(19) United States
(12) Patent Application Publication
(10) Pub. No.: US 2003/0212033 A1
(43) Pub. Date: Nov. 13, 2003

(54) Weight reduction and inhibition of hypertriglyceridemia medicine containing ATP as effective ingredient

(76) Inventor: Chao Song Hsue, Taichung (TW)

Correspondence Address: Chao Song Hsue, P.O. BOX 2103, Taichung, TW

(21) Appl. No.: 10/140,189

(22) Filed: May 8, 2002

(51) Int. Cl. A61K 31/7076; A61K 31/522

(52) U.S. Cl. 514/47; 514/263.32

(57) ABSTRACT

A weight reduction and inhibition of Hypertriglyceridemia medicine contain ATP as an effective ingredient, and more particularly to one that takes advantage of a cyclic adenosine monophosphate, Cyclic AMP) produced from the decomposed ATP in human body to promote the increase of lipase, thus to activate the metabolism and decomposition of triglyceride in the human body.

### BACKGROUND OF THE INVENTION

The present invention is related to a weight reduction and inhibition of Hypertriglyceridemia medicine, and more particularly to one that has ATP as its effective ingredient.

Whereas, as far as the bio-chemical reaction in concerned, Adenosine triphoshate (ATP) is crucial to metabolism of energy and phosphoric acid in biotic organism. As it is decomposed, ATP has its phosphoric group transferred to and activate other substance while producing adenosine-3′-5′-mono phosphoric acid due to reaction of adenyl cyclase, a substance related to hormone. Therefore, ATP is usually used to cure acute and sudden attack of paroxysmal tachycardia. Furthermore, for any other medicines containing ATP are also used to alleviate neuropathy, such as neuritis, and Trigeminal neuralgia.

However, given with its critical role in synthesis and decomposition with other live substances as well as in metabolisms with biological mechanism, adenosine certainly is not restricted to those purposes as described in the preceding paragraph. ATP, an important adenosine can be more widely applied to contribute more in promoting human health.

### SUMMARY OF THE INVENTION

The primary purpose of the present invention is to provide a weight reduction and inhibition of Hypertriglyceridemia medicine containing ATP to promote certain biologic enzymes, thus to encourage metabolism for weight reduction and breakdown of Triglyceride. To achieve the purpose, the present invention contains ATP as its effective ingredient takes advantage of a cyclic adenosine monophosphate, Cyclic AMP) produced from the decomposed ATP in Human body to promote the synthesis of lipase, thus to activate the metabolism and decomposition of Triglyceride in the Human body.

### BRIEF DESCRIPTION OF THE DRAWINGS

FIG. 1 is a view showing a chemical structure formula of ATP.

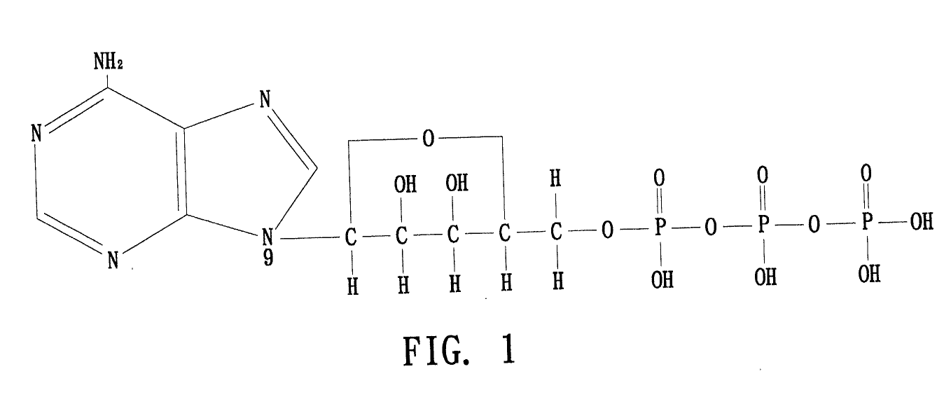

FIG. 2 is a view showing a chemical structure formula of Cyclic AMP.

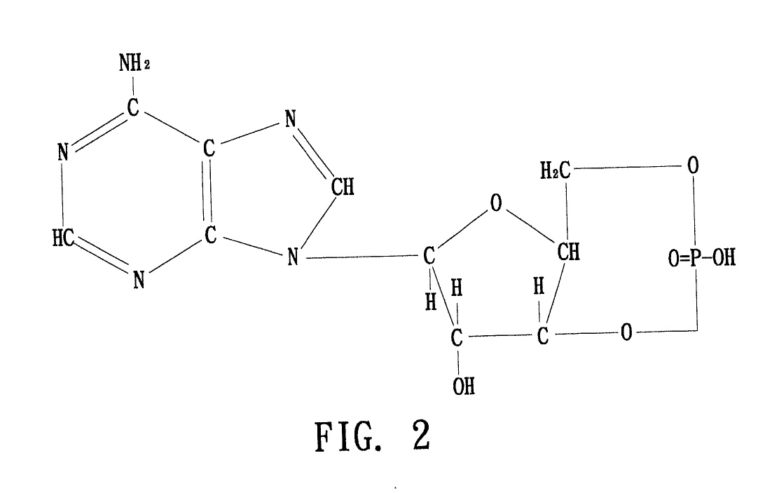

FIG. 3 is a schematic view showing biochemical reactions of the present invention.

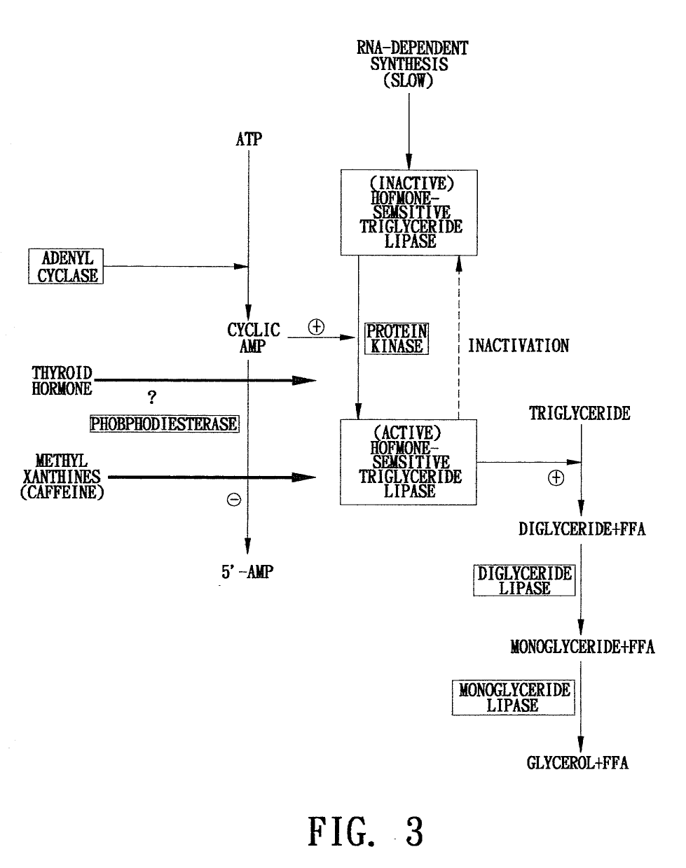

### DETAILED DESCRIPTION OF THE PREFERRED EMBODIMENTS

A weight reduction medicine and lysis of TG (Triglyceride) of the present invention contains ATP as its effective ingredient. ATP being subject to reaction of adenyl clyase produces a cyclic AMP; and the cyclic AMP in turn activates protein kinase to further encourage and activate lipase in Human body. Accordingly, FAT, Adipose tissue and TG can be break into diglyceride and free fatty acid, diglyceride then decomposed into monoglycride and free fatty acid FFA, monoglyceride is then decomposed into glycrol and free fatty acid FFA. As a result, TG has been breaking down into glyceride and FFA which can be further break into H₂O, Co₂ and engery. Not only provides the energy that Human body need, also achieve the purpose of weight reduction and break down of TG.

It is to be noted that in the composition of the present invention, a proper inhibitors may be added to maintain a certain transfer rate for ATP into Cyclic AMP so as to prevent subsequent reaction of the Cyclic AMP to adenosine-5′-monophosphate. Those inhibitors are required to contain sufficient chemical properties to inhibit the Cyclic AMP from producing adenosine-5′-monophosphate. At least one from xanthine derivatives may be selected as the inhibitor. If the xanthine derivatives are selected, any of a 1,3,7 trimethyl-xanthine, 1,3 dimethyl-xanthine or a 3.7 dimethyl-xanthine derivatives or any of their derivatives may be options and any of the option is capable of providing inhibition results sufficient to maintain good transfer rate for ATP to produce the Cyclic AMP, thus to ensure of optimal activation for the lipase. In pharmacological sense, the working mechanism of the present invention is totally different from any weight reduction medicines, either National Health Department approved or USA FDA approved, generally available in the market there are three types of weight reduction medicines in the market, respectively,

1. P.P.A. (Phenylpropanolamine), related to a sympathetic nerve stimulant essentially by affecting the hypothalamus nerve to create lack of appetite and frustrate the desire for food so as to cut down the amount of food taking, thus to achieve the purpose of weight reduction;

2. Xanical (Orlistat), related to an inhibitor for the lipase from the pancreas in the digestion duct by preventing fat in food from being decomposed and absorbed by the human body and thus to have the fat to be forthwith discharged out of the human body; furthermore, the oily excrement is common; and

3. Reductil (Subutramine), related to an inhibitor to the absorption of serum serotonin and noradrenaline, both conduction substances of the nerves system, functioning by developing the sense of SATIETY and thermogenesis.

As disclosed, the present invention achieves its purpose of weight reduction and decomposition of TG by promoting activation and increase of lipase to facilitate decomposition and metabolism of Triglyceride for removing fat accumulated in human body; instead of inhibiting the normal operation of mechanisms in human body. The present invention is totally different than any of the prior art either in pharmacology or mechanical transfer, and creates comparatively less side effects to the human body.

What is claimed is:

1. A weight reduction and inhibition of Hypertriglyceridemia medicine, and more particularly to one that has Adenosine triphoshate (ATP) as its effective ingredient.

2. The weight reduction and inhibition of Hypertriglyceridemia medicine, and more particularly to one that has ATP as its effective ingredient as claimed in claim 1, wherein, it contains at least one inhibitor.

3. The weight reduction and inhibition of Hypertriglyceridemia medicine, and more particularly to one that has ATP as its effective ingredient as claimed in claim 2, wherein, the inhibitor relates to xanthine derivatives.

4. The weight reduction and inhibition of Hypertriglyceridemia medicine, and more particularly to one that has ATP as its effective ingredient as claimed in claim 3, wherein, the inhibitor relates to one selected from a 1,3,7 trimethyl-xanthine, 1,3 dimethyl-xanthine or a 3.7 dimethyl-xanthine derivatives.
